(19) United States
(12) Patent Application Publication
(10) Pub. No.: US 2004/0024533 A1
(43) Pub. Date: Feb. 5, 2004

(54) Method for node mapping, network visualizing and screening

(75) Inventor: Yoshihiro Ohta, Tokyo (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/346,871

(22) Filed: Jan. 21, 2003

(30) Foreign Application Priority Data
Aug. 5, 2002 (JP) 2002-227418

(51) Int. Cl. G01V 1/40; G06F 19/00; G01N 33/48; G01N 33/50; G01N 15/08

(52) U.S. Cl. 702/19

(57) ABSTRACT

An automated method for creating easily viewable network visualizations without direct use involvement. A table which includes as elements node types, the number of connecting nodes to be connected to the nodes, and the number of end nodes to be connected to the nodes, is prepared by searching databases that stores interactions between nodes, and connecting nodes that are connected to a predetermined number of, or more, end nodes are extracted from this table. The extracted connecting nodes are arranged onto a visualization space at a distance of not less than a preset distance, and the remaining connecting nodes are arranged onto the visualization space. Thereafter, the arrangement of the end nodes in the visualization space is computed, and the distance between the connecting nodes is adjusted so that the end nodes do not overlap.

### BACKGROUND OF THE INVENTION

1. Field of the Invention

The present invention relates to a technology to display a network of interacting proteins or genes, DNA, or the like, and in particular to a method of node mapping for network visualization, a method for network visualization, and a method for screening.

2. Description of the Related Art

With the progress of human genome projects, there is an increasing demand for the function analysis of proteins which are coded on obtained DNA sequences. The functions of proteins are featured by interactions with other materials, and thus attempts for encyclopedic determination on interactions are being undertaken vigorously. Meanwhile, other attempts to obtain interaction information from the literature have been started. To visualize large quantities of the obtained interaction information in an easily understandable manner is very important for correct interpretation of the interaction information.

One of the methods for visualizing interaction information on proteins, etc. is a visualization method in the form of a network wherein materials are linked with line segments. Typical examples thereof are available at Myriad online (HYPERLINK “URL::www.myriad.com/online/” URL::www.myriad.com/online/). This visualization method of network form is suitable to visualize chain linkages of interaction information.

According to conventional visualization methods of network form, when networks having nodes of DNA, genes and proteins are drawn, the nodes are arranged at random. Therefore, if there is difficulty in viewing the visualized network, a user would have to appropriately re-arrange the nodes by himself. This method can be used for up to approximately several dozens of nodes without any problems. However, when there are more nodes, linkage lines between nodes in the display becomes too complex to be viewed, thereby making it impossible to understand the network. Further, in these conventional methods, a network is projected only onto a two-dimensional plane, and thus it is impossible to visualize and therefor understand reflect the properties of the network based on the arrangement of, for example, three-dimensional periodical boundary conditions.

In view of the present situation for network visualization on the interactions between materials, it is an object of the present invention to provide a node mapping method for easily viewable automated network visualizations without the user's direct involvement, a network visualization method, and a screening method.

### SUMMARY OF THE INVENTION

As a method for arranging individual nodes in a network in order to make easily viewable network visualizations, a method wherein nodes are arranged at random and then re-arranged into a highly symmetric arrangement is considered. This method is theoretically possible by assuming proper potential between nodes, but it is not practical because a good amount of time is required for computation. Beyond that, computation time will be enormous for handling networks having several thousands of, or more, nodes combined, and it will thus be substantially impossible to draw these networks. Therefore, according to the present invention, there is provided a method for arranging nodes with high symmetry from the start. Nodes are arranged in consideration of symmetry. Thus, even though nodes represent not single proteins but conjugated proteins having several proteins conjugated, a conjugate is handled as one node. Alternatively, visualization is available in consideration of the symmetry of conjugate by a function to allocate proteins as constituent elements of the conjugate to each node.

A node mapping method for network visualization according to the present invention comprises the steps of:

searching a database that stores interaction between nodes, and preparing a table which includes as elements node types, the number of connecting nodes to be connected thereto, and the number of end nodes to be connected thereto;

extracting from the table connecting nodes that are connected to a predetermined number of, or more, end nodes;

arranging the extracted nodes onto a visualization space at a certain distance from each other, wherein the distance is not less a predetermined distance in accordance with the number of connecting nodes existing therebetween;

arranging the remaining connecting nodes onto the visualization space;

computing the arrangement of the end nodes on the visualization space; and

adjusting the distance between the connecting nodes so that the end nodes do not overlap.

Herein, the phrase “connecting node” means a node having not less than two bonds and the phrase “end node” means a node having one bond.

The network visualization method of the present invention is featured by visualizing on the screen line segments which represent the connection between connecting nodes as well as node mapping in the above manner and visualizing nodes onscreen according to the node mapping.

The nodes typically represent proteins. In addition, the visualization space is typically a two-dimensional regular lattice.

A method for screening a regulatory substance according to the present invention comprises the steps of: extracting an interaction between nodes to be noted from the network visualized on the screen as described above; and screening the regulatory substance which regulates the interaction. The regulatory substance is a substance which facilitates or attenuates the interaction.

### BRIEF DESCRIPTION OF THE DRAWINGS

FIG. 1 is a schematic view of an onscreen network visualization system according to the present invention.

FIG. 2 is a flow chart illustrating an example process for a network visualization-processing unit.

FIG. 3 is a flow chart illustrating one example on how to arrange connecting nodes.

FIG. 4 is a view illustrating an example visualization of a pathway.

FIG. 5 is a view which describes mapping fundamental connecting nodes on a tetragonal lattice.

FIG. 6 is a view which describes mapping connecting nodes on a tetragonal lattice.

FIG. 7 is a view illustrating an example of pathway visualization.

FIG. 8 is a view illustrating an example of pathway visualization.

FIGS. 9A to 9C are views illustrating examples of regular lattices on a two-dimensional plane.

FIG. 10 is a view illustrating a three-dimensional regular polyhedron packed with spheres.

FIG. 11 is a view illustrating a three-dimensional tetragonal lattice.

FIG. 12 is a view illustrating a state wherein grids are equidistantly drawn on a surface-of a cylinder type.

FIG. 13 is a view illustrating a state wherein network visualization is made on a curved surface with rugates.

### PREFERRED EMBODIMENTS OF THE PRESENT INVENTION

Hereinafter, embodiments of the present invention will be described with reference to the drawings. Although proteins are used here as examples to describe a method for creating pathways, the present invention is applicable to other materials such as genes and DNA. Further, when conjugated proteins are decomposed into protein groups and the relationships of the proteins among the protein groups are visualized, it is possible to draw the relationships on a two- or three-dimensional space in the same manner as drawing pathways based on binary relations between single proteins.

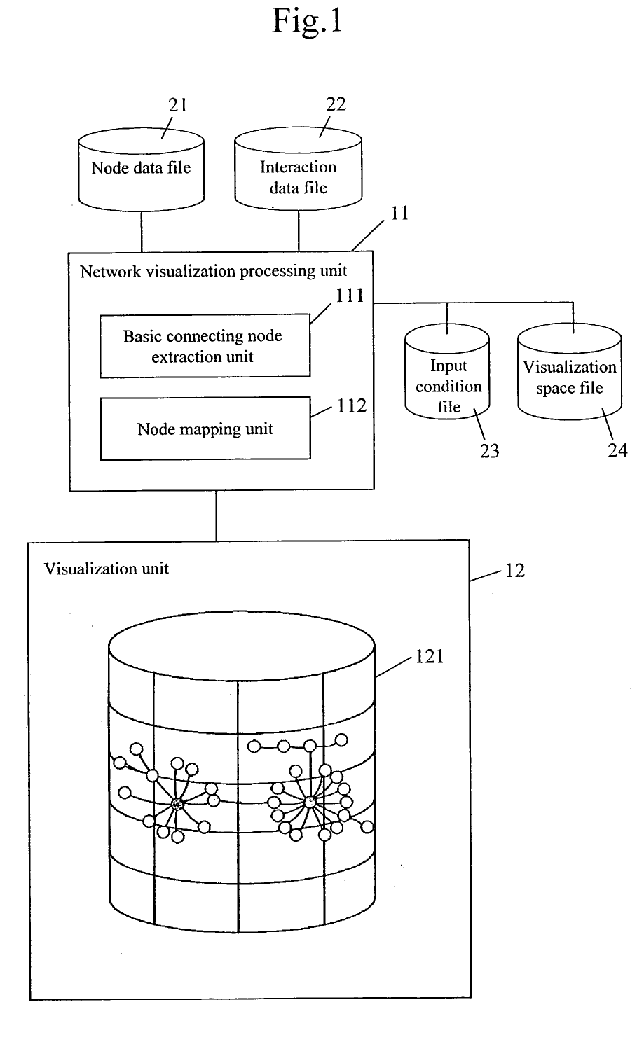

FIG. 1 is a schematic view illustrating an onscreen network visualization system according to the present invention. Described herein is a case whereby names of single or conjugated proteins are used as nodes.

A network visualization processing unit 11 is connected to a node data file 21, an interaction data file 22, an input condition file 23, a visualization space file 24, and a visualization unit 12. The node data file 21 stores the protein names and their types, and property data of proteins such as single proteins or conjugate proteins. The interaction data file 22 stores data showing whether there is an interaction between two randomly chosen proteins (nodes), that is an interaction relationship between the nodes. The node data file 21 and interaction data file 22 are typically created by searching databases for interaction relationship information between proteins. Moreover, they may be created by collecting interaction information from experiments or literature searches. Among the obtained information on interactions between proteins, information concerning proteins is stored as node data in the node data file 21 and information concerning interactions are stored in the interaction data file 22.

The visualization space file 24 stores various lattice point data such as spaces for mapping nodes and pathways, and a tiling method therefor. For example, lattice point data on two-dimensional tetragonal lattice, lattice point data on various curvature surfaces, lattice point data on complicated arabesque, and the like are stored. A user can decide which lattice point data stored in the visualization space file 24 should be used for mapping. The input condition file 23 is, a file in which property conditions for drawing such as dimensions of visualization space (two or three dimension), numbers of visualized nodes, and distances between lattice points are written. Further, the input condition file 23 allows for the selection between a time-dependent changed image indicating time-series changes and a stationary image, or between an instantaneous image and an average image. Furthermore, the user designates the maximum number of nodes, that is, how many nodes should be contained for drawing a network. Moreover, the minimum distance to be maintained between lattice points as the distance between nodes is inputted.

The network visualization processing unit 11 comprises a fundamental connecting node extraction unit 111 which extracts fundamental connecting nodes from the interaction data file 22 between nodes, and a node mapping unit 112 which performs computation for mapping nodes onto the visualization space. The node mapping unit 112 arranges nodes on lattice points in a visualization space designated from the visualization space file by the method described below, in accordance with conditions designated by the input condition file. The visualization unit 12 displays the obtained network information between proteins. In the figure, the visualization unit 12 displays an example of network visualization wherein a visualization space has a cylinder surface and protein nodes are arranged on lattice points equidistantly set on the cylinder surface.

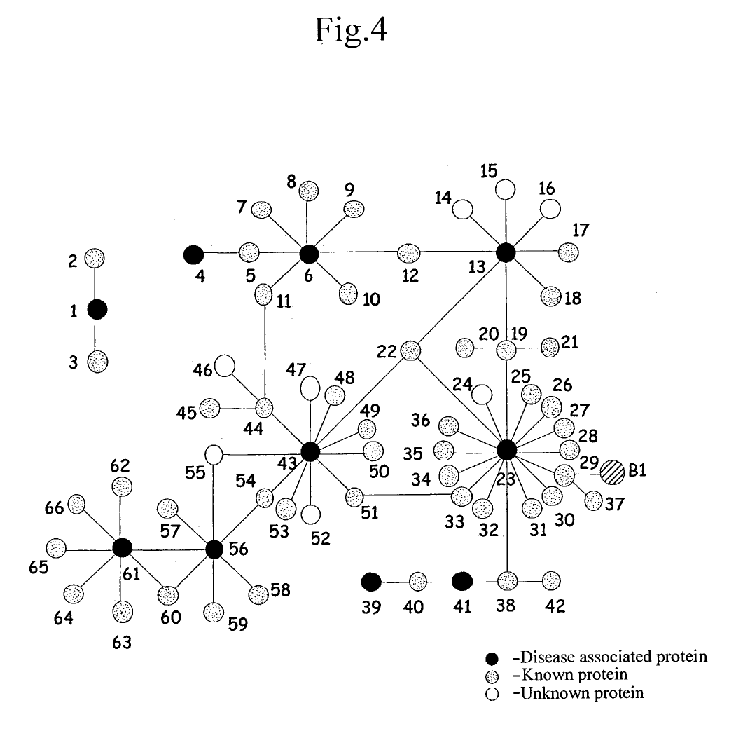

Here, how to map proteins on a two-dimensional tetragonal lattice is described, taking pathways described in FIG. 4 as examples. To make the description easy to understand, it is assumed that pathways could be drawn in advance and the numbering carried out, and then how to draw the pathways is described hereinafter. However, in computing the pathways, as long as the relationships between the nodes are known, it is possible to automatically compute them in the same way as in Table 1 shown below. Thus, even though it is not assumed that the pathways are drawn in advance, the central algorithm of the present invention is valid. Further, the mapping of pathways on a tetragonal lattice is used herein as an example. However, in mapping the other space according to the algorithm of the present invention, projected relationships between one space and the other space are used, or a space is defined in advance for creating a network. Therefore, it is possible to easily draw pathways in other dimensional space or on other lattice points. Here, it is assumed that lattice intervals are accurately defined. However, even if the arrangement of the lattice points are at random, the node arrangement can be determined with reference to the distance, as long as the distance between lattice points is defined. Thus, it is possible to symmetrically arrange the nodes when the distance concerning lattice points is defined. When the visualization space is a curved face such as a sphere or a cylindrical face, geodesic lines are used in order to measure the distances.

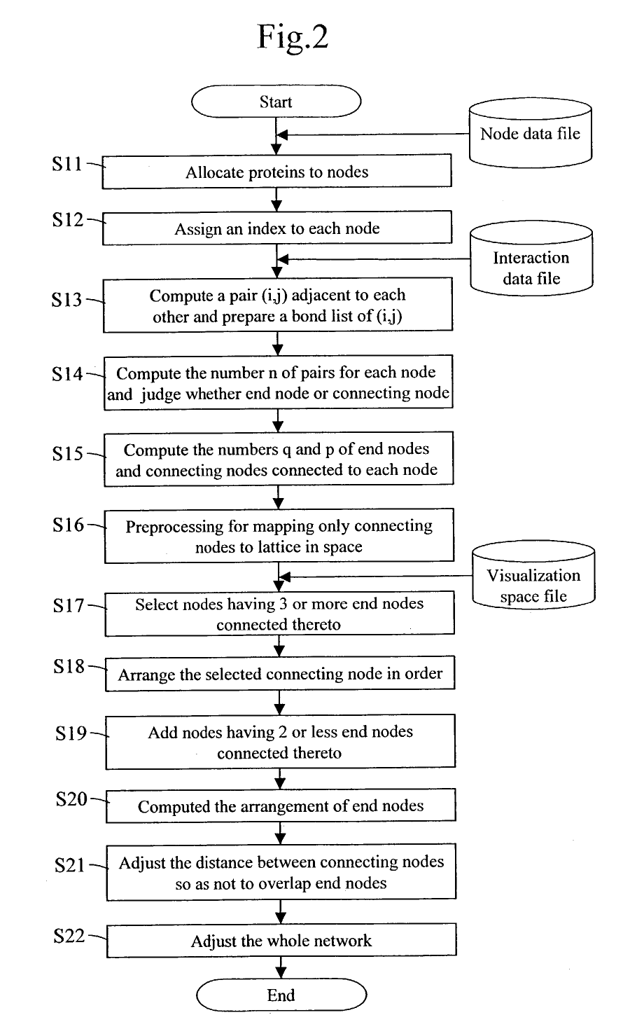

FIG. 2 is a flow chart illustrating an example process for arranging the nodes with high symmetry in the network visualization processing unit 11.

First, the node data file 21 is read in, and individual proteins stored therein are allocated to nodes. Then, investigation is made on node properties and whether the proteins are conjugated or single (Step 11). Next, an index i is given to each node in accordance with the node data file 21. Each node is handled as a single node at first for giving an index, and only in the case of conjugated nodes, further indexes are additionally given thereto by the number of nodes (Step 12).

Next, the interaction data file 22 is read in, and an adjacent node j connected to the node i is computed to make a pair of (i, j). Then, a bond list indicating the interaction between indexes i and j is prepared (Step 13). For individual nodes, the number n of adjacent nodes (the number of bonds) to be paired with the individual nodes is computed, and it is judged whether the adjacent nodes are an end node which is connected to no nodes or a connecting node which is connected to other node (Step 14). Then, for individual nodes, the number q of end nodes connected thereto and the number p of connecting nodes connected thereto are computed (Step 15). In this process, prepared and stored in the system is a table, like Table 1 described below, which keeps on record the interactive relationships, the number n of adjacent nodes, the number p of connecting nodes, and the number q of end nodes for individual indexes. In Table 1, the expression “i-j” means that the node with index i is connected to the node with index j. Also, n is equal to the sum of p and q. When a node like a node 29 in FIG. 4 has a bond yet no node information, it is regarded as a boundary node B1 and is handled as a node.

TABLE 1

| | | Number | | |
|---|---|---|---|---|
| | | of | | |
| | | adjacent | Number of | Number of |
| | | nodes | connecting | end |
| i | i-j pair ( 1 ≦ j ) | (n) | nodes (p) | nodes (q) |
| 1 | 1-2, 1-3 | 2 | 0 | 2 |
| 2 | 2-1 | 1 | 1 | 0 |
| 3 | 3-1 | 1 | 1 | 0 |
| 4 | 4-5 | 1 | 1 | 0 |
| 5 | 5-4, 5-6 | 2 | 1 | 1 |
| 6 | 6-5, 6-7, 6-8, 6-9, 6-10, 6-11, 6-12 | 7 | 3 | 4 |
| 7 | 7-6 | 1 | 1 | 0 |
| 8 | 8-6 | 1 | 1 | 0 |
| 9 | 9-6 | 1 | 1 | 0 |
| 10 | 10-6 | 1 | 1 | 0 |
| 11 | 11-6, 11-44 | 2 | 2 | 0 |
| 12 | 12-6, 12-13 | 2 | 2 | 0 |
| 13 | 13-12, 13-14, 13-15, 13-16, 13-17, | 8 | 3 | 5 |
| | 13-18, 13-19, 13-22 | | | |
| 14 | 14-13 | 1 | 1 | 0 |
| 15 | 15-13 | 1 | 1 | 0 |
| 16 | 16-13 | 1 | 1 | 0 |
| 17 | 17-13 | 1 | 1 | 0 |
| 18 | 18-13 | 1 | 1 | 0 |
| 19 | 19-13, 19-20, 19-21, 19-23 | 4 | 2 | 2 |
| 20 | 20-19 | 1 | 1 | 0 |
| 21 | 21-19 | 1 | 1 | 0 |
| 22 | 22-13, 22-23, 22-43 | 3 | 3 | 0 |
| 23 | 23-19, 23-22, 23-24, 23-25, 23-26, | 16 | 5 | 11 |
| | 23-27, 23-28, 23-29, 23-30, 23-31, | | | |
| | 23-32, 23-33, 23-34, 23-35, 23-36, | | | |
| | 23-38 | | | |
| 24 | 24-23 | 1 | 1 | 0 |
| 25 | 25-23 | 1 | 1 | 1 |
| 26 | 26-23 | 1 | 1 | 1 |
| 27 | 27-23 | 1 | 1 | 1 |
| 28 | 28-23 | 1 | 1 | 1 |
| 29 | 29-23, 29-37, 29-B1 | 3 | 1 | 2 |
| 30 | 30-23 | 1 | 1 | 0 |
| 31 | 31-23 | 1 | 1 | 0 |
| 32 | 32-23 | 1 | 1 | 0 |
| 33 | 33-23, 33-51 | 2 | 2 | 0 |
| 34 | 34-23 | 1 | 1 | 0 |
| 35 | 35-23 | 1 | 1 | 0 |
| 36 | 36-23 | 1 | 1 | 0 |
| 37 | 37-29 | 1 | 1 | 0 |
| 38 | 38-23, 38-42, 38-41 | 3 | 2 | 1 |
| 39 | 39-40 | 1 | 1 | 0 |
| 40 | 40-39, 40-41 | 2 | 1 | 1 |
| 41 | 41-40, 41-38 | 2 | 2 | 0 |
| 42 | 42-38 | 1 | 1 | 0 |
| 43 | 43-22, 43-44, 43-47, 43-48, 43-49, | 11 | 5 | 6 |
| | 43-50, 43-51, 43-52, 43-53, 43-54, | | | |
| | 43-55 | | | |
| 44 | 44-11, 44-43, 44-45, 44-46 | 4 | 2 | 2 |
| 45 | 45-44 | 1 | 1 | 0 |
| 46 | 46-44 | 1 | 1 | 0 |
| 47 | 47-43 | 1 | 1 | 0 |
| 48 | 48-43 | 1 | 1 | 0 |
| 49 | 49-43 | 1 | 1 | 0 |
| 50 | 50-43 | 1 | 1 | 0 |
| 51 | 51-33, 51-43 | 2 | 2 | 0 |
| 52 | 52-43 | 1 | 1 | 0 |
| 53 | 53-43 | 1 | 1 | 0 |
| 54 | 54-43, 54-56 | 2 | 2 | 0 |
| 55 | 55-43, 55-56 | 2 | 2 | 0 |
| 56 | 56-54, 56-55, 56-57, 56-58, 56-59, | 7 | 4 | 3 |
| | 56-60, 56-61 | | | |
| 57 | 57-56 | 1 | 1 | 0 |
| 58 | 58-56 | 1 | 1 | 0 |
| 59 | 59-56 | 1 | 1 | 0 |
| 60 | 60-56, 60-61 | 2 | 2 | 0 |
| 61 | 61-56, 61-60, 61-63, 61-64, 61-65, | 2 | 2 | 0 |
| | 61-66, 61-62 | | | |
| 62 | 62-61 | 1 | 1 | 0 |
| 63 | 63-61 | 1 | 1 | 0 |
| 64 | 64-61 | 1 | 1 | 0 |
| 65 | 65-61 | 1 | 1 | 0 |
| 66 | 66-61 | 1 | 1 | 0 |
| B1 | B1-29 | 1 | 1 | 0 |

Thereafter, the input condition file 23 is read in, and preprocessing for mapping only the connecting nodes on lattice points in the space is carried out and the quantity of nodes that exist between connecting nodes is computed (Step 16).

Here, a space symmetric file is read in from the visualization space file 24. As an example, a two-dimensional tetragonal lattice is taken herein. When there are too many connecting nodes, it becomes difficult to provide space symmetry for space-mapping and thus, the number of nodes for mapping is delimited. According to this embodiment, in the basic connecting node extraction unit 111, nodes with an end node number q of three or more are selected, and at first, only nodes of conjugated proteins composed of three or more constituent proteins are visualized. In the embodiment shown in Table 1, when nodes with an end node number of three or more are selected, connecting nodes with indexes 6, 13, 23, 43, 56, and 61 are picked up for visualization (Step 17). Then, the selected connecting nodes are arranged accordingly (Step 18).

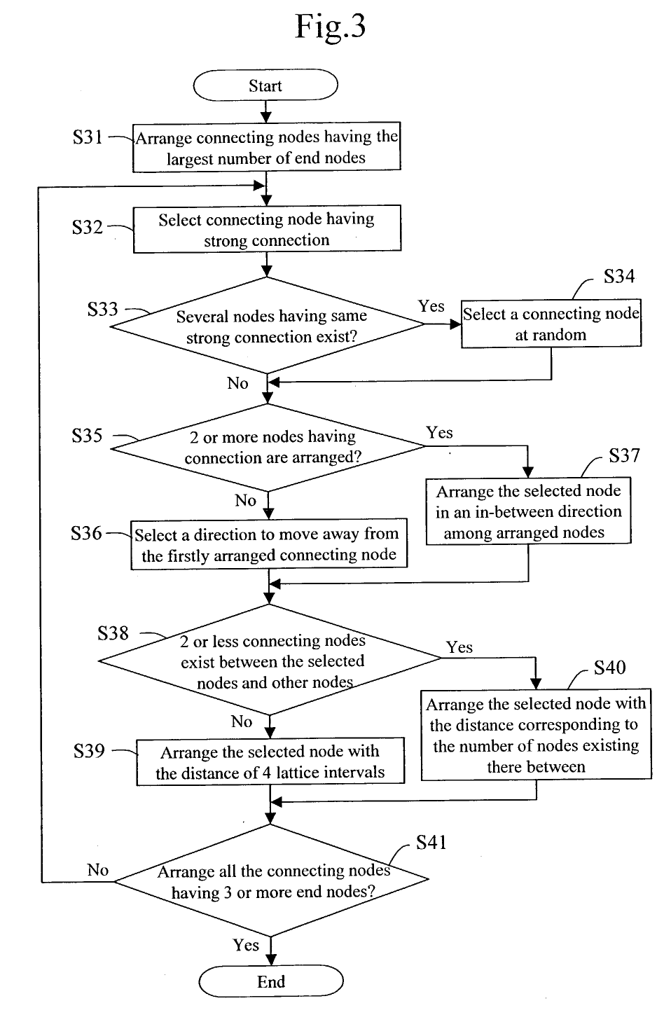

FIG. 3 is a flow chart illustrating one example of the process of Step 18 in detail. The selected connecting nodes are arranged in order from a connecting node having the largest number of end nodes (Step 31), to a node having strong connection with the connecting node (that is a connecting node having fewer connecting nodes therebetween) (Step 32). When there are several connecting nodes having the same strong connection (response at Step 33 is “Yes”), one connecting node is selected at random from these connecting nodes having the same strong connection (Step 34) and further another connecting node having strong connection with the selected connecting node is selected. This process is repeated to determine the arrangement order.

Next, according to the determined order, the connecting node is arranged in a proper direction against the previously arranged connecting node group with a proper node interval distance. When the connecting node to be arranged has connection with only one previously arranged connecting node (response at Step 35 is “No”), the connecting node is arranged in a direction to move away from the firstly arranged connecting node (Step 36). When the connecting node to be arranged has connections with two or more previously arranged connecting nodes (response at Step 35 is “Yes”), the connecting node is arranged in an in-between direction among these connecting nodes (Step 37)

After the direction against the group of previously arranged connecting nodes is determined, the distance is properly set for the arrangement. In this embodiment, when the number (the number can be obtained using the preprocessing information) of connecting nodes between a connecting node to be arranged and a previously arranged connecting node to be connected thereto is three or more (response at Step 38 is “No”), the connecting node is arranged on lattice points at a distance of 4 lattice intervals from the previously arranged connecting node (Step 39). When the number is two or less, the connecting node is arranged on lattice points at a lattice interval distance corresponding to the number of connecting nodes existing therebetween (Step 40). For example, when there is no connecting node between a connecting node to be arranged and a previously arranged connecting node to be connected thereto, the connecting node is arranged at one interval distance from the previously arranged connecting node. In this manner, when there are one, two and three or more connecting nodes therebetween, the connecting node is arranged on lattice points at a distance of two, three and four lattice intervals, respectively. It is noted that the distance mentioned herein is a minimum distance to be kept therebetween, and thus they may be arranged at greater distances. The above process is repeated until all the selected nodes are arranged.

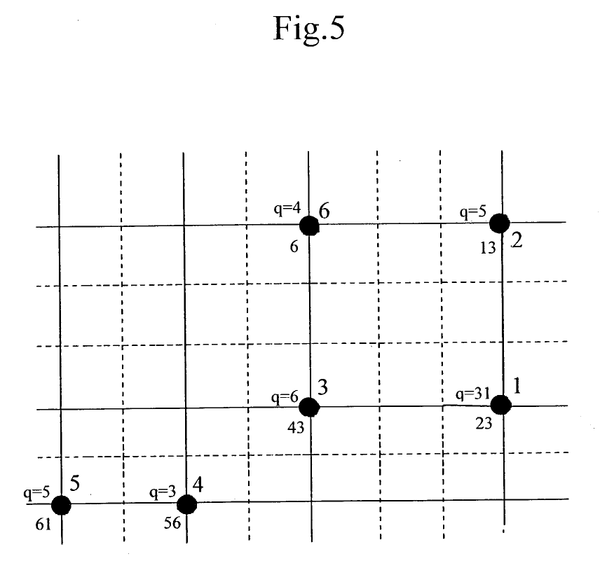

In the embodiment shown in FIG. 4, a connecting node with index 23 having the largest number of end nodes is first arranged, and then, using information of the preprocessing, connecting nodes with indexes 13 and 43 having strong connection with the connecting node with index 23 are randomly arranged on lattice points at a distance of three lattice intervals. Next, a connecting node with index 43 and connecting nodes with indexes 56 and 61 having smaller connecting node number therebetween, in this order, are randomly arranged in the opposite to the connecting node with index 23 direction (the direction away from connecting node with index 23). Finally, a connecting node with index 6 is arranged between the connecting nodes with indexes 13 and 43. The results are shown in FIG. 5.

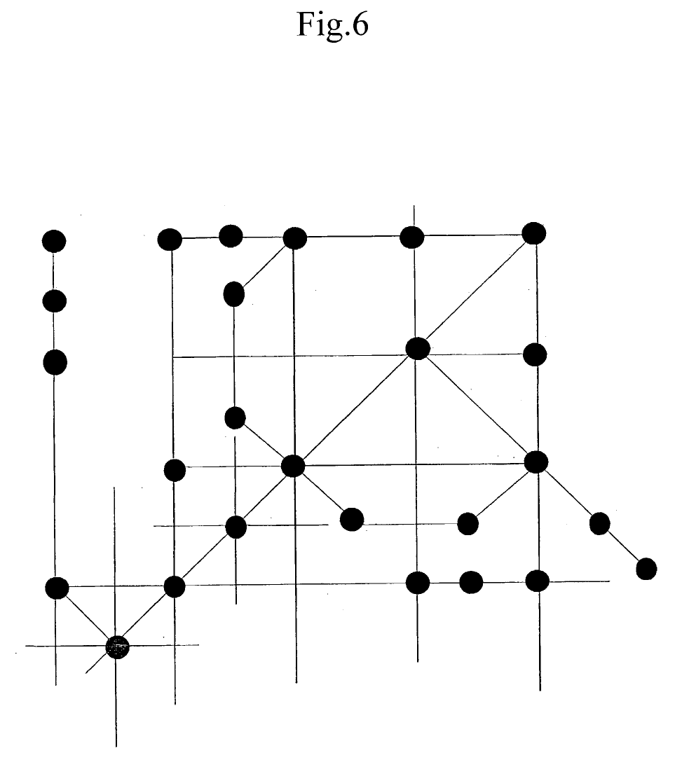

Next, connecting nodes having the end node number of less than 3 are selected and arranged on lattice points. At this time, while giving attention to the connection between connecting nodes, they are arranged on lattice points (Step 19). The results are shown in FIG. 6, which illustrates all the connecting nodes. In FIG. 6, some end nodes are visualized to make the figure easy to understand. Then, on the basis of the arrangement of these connecting nodes, the computation on the arrangement of end nodes is carried out (Step 20). At this time, the computation is carried out so that the end nodes are arranged as evenly as possible. Thereafter, the distances between the connecting nodes are adjusted so as not to overlap the end nodes (Step 21). Lastly, the whole network is adjusted (Step 22). To adjust the whole network, for example, the distance potential between the nodes is presumed, and the node arrangement is computed so as to keep sufficient distance among the whole nodes including end nodes and connecting nodes. Here, it is premised that, for example, a strong potential is applied on the lattice interval distance of 1.5 or more, and no potential is applied on the lattice interval distance of less than 1.5, the final result are shown in FIG. 4. The process for mapping of and the adjustment process for arranging the nodes are carried out in the node mapping unit 112.

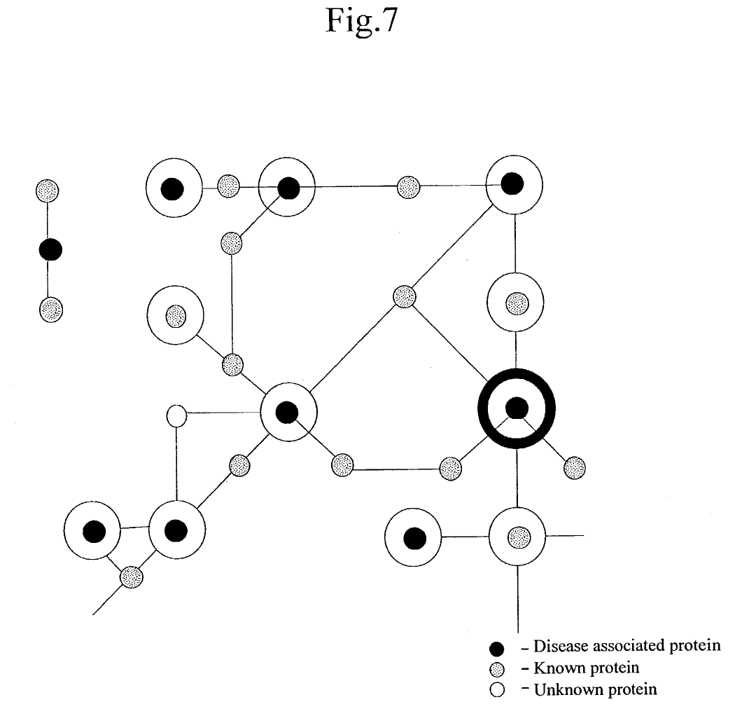

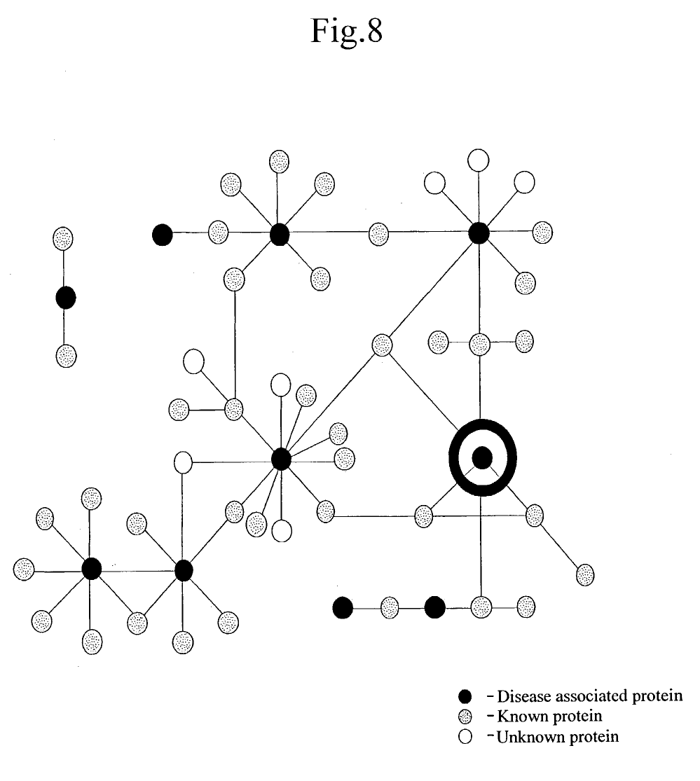

In addition, the relationships between connecting nodes and end nodes are freely changeable, and therefore various combinatorial visualizations of end nodes and connecting nodes is available as shown in FIGS. 7 and 8. FIG. 7 shows a format wherein almost all the end nodes are omitted. FIG. 8 shows a case wherein all the visualized nodes are end nodes except that some of them are connecting nodes. Further, in the case of conjugated proteins, visualization with graphical formula frames or sphere arrangement in graphical formula is available.

The visualization space file 24 maintains various lattice point data concerning spaces for mapping pathways such as regular lattice and complicated arabesque, and a tiling method therefor. The format of geometric data is generally a format wherein a fundamental vector is associated with each figure. In the case of three-dimensional curved surface, coordinate vector data composed of polar coordinates for each figure using may be maintained. Meanwhile, in order to clearly distinguish individual figures in a three-dimensional space, the values of space filling factors, branch direction, branch angle, face direction or the like are used for defining a space, as described in, for example, Peter Pearce, “Structure in Nature is a Strategy for Design” MIT Press, 1990, pp.72-73, 76-77, 82-83, 96-103, 108-115, 152-153.

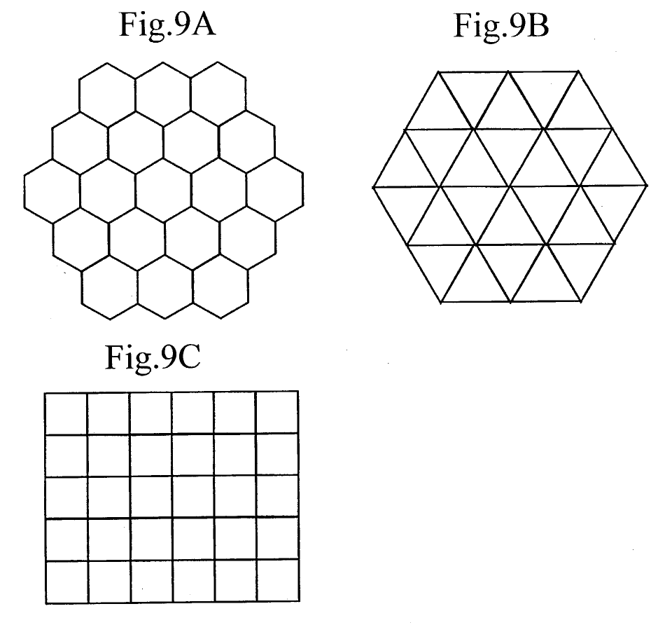

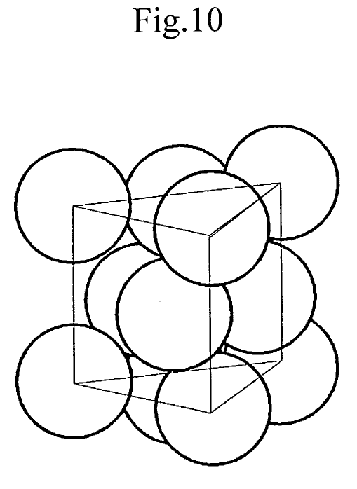

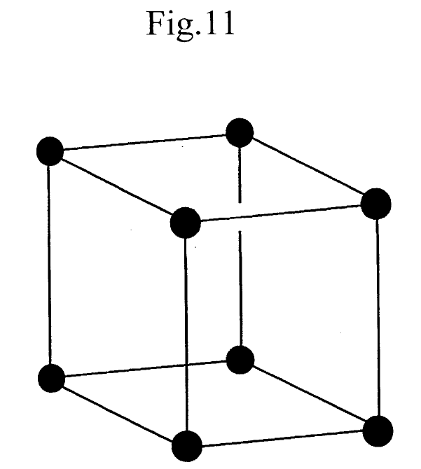

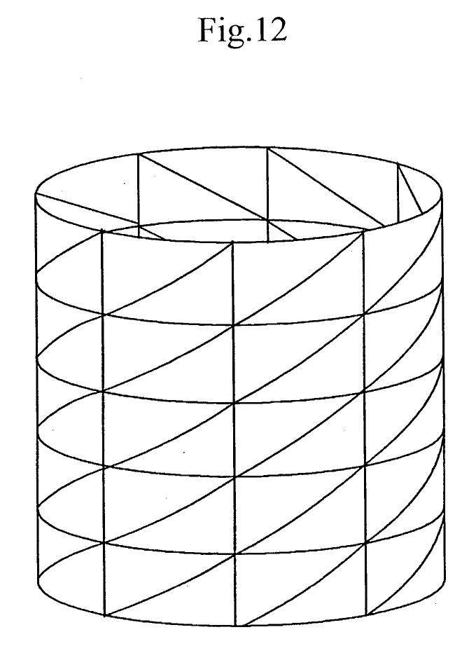

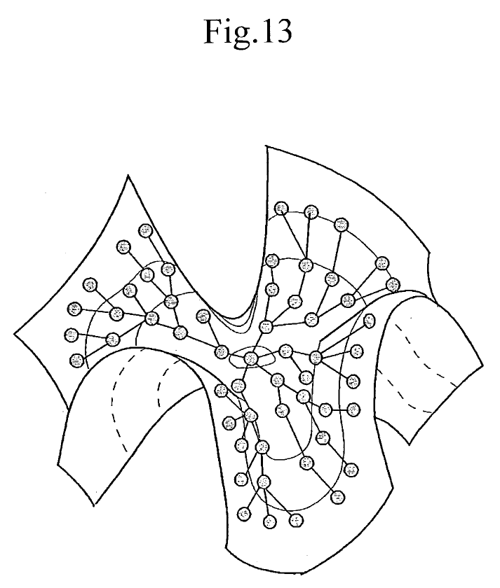

Here, some of the space figures are described. FIGS. 9A to 9C are views illustrating examples of a regular lattice on a two-dimensional plane. Protein nodes are arranged on these lattice points and a network is visualized. FIG. 10 is a view illustrating a three-dimensional regular polyhedron packed with spheres. FIG. 11 is a view illustrating a three-dimensional tetragonal lattice. When protein nodes are arranged on these lattice points, a three-dimensional network is visualized. FIG. 12 is a view illustrating a state wherein grids are equidistantly drawn on the surface of a cylinder. Protein nodes can be arranged on a surface of a cylinder like this, and thereby a network can be visualized on a polyhedron. FIG. 13 is a view illustrating a state wherein network visualization is made on a curved surface with rugates. Even when nodes are densely present, they can be visualized without overlapping by increasing the depth of rimples and enlarging a surface area.

The above space figures are effective when the pathways can be handled as an isolated system or are periodic. When some of the pathways are periodic, it is easy to understand the network by mapping these pathways on a torus or a curved surface having geometric directivity such as a spiral and a hypersurface. This allows for visualization in an easily visible form in cases of complicated boundary conditions. The visualization of this type is effective since it is possible to express the network in an easily visible form when a node has many bonds particularly at a center point of a hypersurface.

Heretofore, proteins are taken as examples for the explanation, but other biological substances such as DNA, or individuals of strains of family analysis may be used as nodes for network visualization. In particular, when conjugated proteins are degraded into protein groups and the relationships of proteins among the protein groups is visualized, it is possible to visualize a network in a two- or three-dimensional space in the same manner as drawing pathways on the basis of binary relationships between single proteins.

According to the network visualization of the present invention, it is possible to avoid the viewing difficulty caused by the overlapping of nodes, and thus a user hardly overlooks interactions between proteins. The user can extract the interaction between noteworthy proteins from this network visualization and conduct screening tests on a regulatory substance which regulates the interaction.

For example, test compounds are subjected to in vitro screening tests for identifying a compound having binding ability to a protein conjugate or a protein member to be interacted with the protein conjugate, both of which are deduced from the network visualization. To this end, a specific interaction between the test compounds and target components, that is the protein conjugates or the protein members to be interacted with the protein conjugates. Then, they are reacted with each other for a sufficient time under sufficient conditions which allow conjugates to be purified by binding the compounds to the target components. Thereafter, the binding is detected. This screening enables the identification of an agonist which is a compound that enhances activities or properties desirable for protein interaction, or an antagonist which is a compound that interferes or inhibits activities or properties desirable for protein interaction.

As screening methods, various known methods can be employed. Protein conjugates and protein members to be interacted therewith can be prepared by appropriate methods such as recombinant expression and purification. Protein conjugates and/or protein members to be interacted therewith (herein both are referred to as “targets”) may be dissolved in a free state. Test compounds may be mixed with the targets thereby to prepare a liquid mixture. Test compounds may be labeled with detectable markers. Under proper conditions, conjugates containing the targets are bound to and co-immunoprecipitated with the test compounds, and then washed. The test compounds in the precipitated conjugates can be detected because of the markers attached to them.

In a preferable embodiment, the targets may be fixed on a solid supporting body or cell surface. Preferably, the targets can be arranged in an array so as to prepare a protein microchip. For example, the targets may directly be fixed onto a microchip substrate, like a slide glass, or a multi-well plate with nonneutralizing antibodies, that is antibodies which have the ability to bind with the targets but do not cause substantial damage on the biological activity of the targets. For screening, the test compounds are brought into contact with the fixed targets and are bound to the targets under standard test conditions for binding, thereby producing conjugates. Either the targets or the test compounds are labeled with detectable marker by using known labeling techniques. For example, U.S. Pat. No. 5,741,713 discloses combinatorial libraries of biochemical compounds labeled with NMR active isotopes. In order to identify compounds to be bound thereto, the production of conjugates produced from the targets and test compounds, or the kinetics of their production may be measured. When screening organic non-peptides or non-nucleic acid compounds, it is preferable to use labeled or coded (namely “labeled”) combinatorial libraries so as to swiftly decode a lead structure. The reason why this is particularly important is that individual compounds observed in chemical libraries are not self-amplified. Labeled combinatorial libraries are described, for example, in Borchardt and Still, J. Am. Chem. Soc., 116: 373-374 (1994) and Moran et al., J. Am. Chem. Soc., 117: 10787-10788 (1995).

On the contrary, for example, the test compounds may be fixed on a solid supporting body thereby preparing a micro array of the test compounds. Then, the target protein or protein conjugates are brought into contact with the test compounds. The targets may be labeled with detectable markers. For example, before the binding reaction, the targets can be labeled with radioisotopes or fluorescent markers. Alternatively, after the binding reaction, bound targets are detected by using: antibodies which are immunoreactive to the target and are labeled with radioactive substances, fluorescent markers, enzymes or the like; or labeled anti-immunoglobulin secondary antibodies, resulting in the identification of the compounds binding therewith. A protein probing method is one example of accomplishing this. Namely, the targets are used as probes for screening protein expression libraries. The expression libraries may be phage display libraries, libraries based on in vitro translation, or ordinary expression cDNA libraries. The libraries may be fixed onto a solid supporting body such as nitrocellulose filter. References may be made to, for example, Sikela and Hahn, Proc. Natl. Acad. Sci. USA, 84: 3038-3042 (1987). The probes may be labeled with a radioisotope or fluorescent marker. Alternatively, the probes may be biotinylated so that they can be detected using streptavidin-alkaline phosphatase conjugates. Further, it is convenient to detect the bound probes using antibodies.

According to another embodiment, competitive binding tests can be conducted using ligands known to have the ability to bind with the targets. The known ligands are reacted with the targets thereby generating conjugates, and the conjugates are brought into contact with the test compounds. The ability of the test compounds to interfere the interaction between the targets and the known ligands is measured. One typical ligand is an antibody which can specifically bind to the target. Antibodies of this type are particularly useful for identifying peptides which have one or more kinds of common epitope with the target protein conjugates or the protein members to be interacted therewith.

According to a specific embodiment, the protein conjugates to be used for the screening test contains 2 kinds of interactive proteins or hybrid proteins which are formed by the fusion of fragments or domains thereof. The hybrid proteins may contain epitope labels fused thereto for detection. Suitable examples of epitope labels of this type include sequences derived from hamagglutinin (HA) of influenza virus, simian virus 5 (V5), poly-histidine (6×His), c-myc, lacZ, GST, or the like.

Further, the test compounds can also be used in in vitro tests for identifying compounds which have the ability to dissociate protein conjugates identified according to the present invention. Therefore, for example, protein conjugates containing protein 1 are brought into contact with the test compounds thereby to detect the protein conjugates. On the contrary, the screening of the test compounds allows for the enhancement of the interaction between protein 1 and proteins to be interacted therewith, or the identification of compounds having the ability to stabilize protein conjugates generated from 2 kinds of proteins.

This test can be carried out in a manner similar to the above binding test. For example, the presence or absence of particular protein conjugates can be determined with antibodies which are selectively immunoreactive with the protein conjugates. Thus, after the protein conjugates are subjected to incubation with the test compounds, immune precipitation test can be conducted using the antibodies. If the protein conjugates are fragmented by the test compounds, the amount of the protein conjugates to be precipitated with immunoreaction in this test would be remarkably smaller than the amount of the control test wherein the protein conjugates are not brought into contact with the test compounds. Likewise, when the interaction between 2 kinds of proteins is to be enhanced, they are subjected to incubation with the test compounds. Thereafter, the protein conjugates can be detected with antibodies having selective immunoreactivity. Namely, comparison in terms of the amount of generated protein conjugates may be made to assess the presence or absence of the test compounds.

According to the present invention, after obtaining necessary binary relationship among genes or proteins from experiments or huge databases, these relationships can effectively be visualized in an easily understandable form. Since network visualization is carried out well-symmetrically in a short period, it is possible to predict thus far unknown binary relationships, on the basis of known binary relationships. This prediction allows for the finding of novel pathways relevant to diseases etc., thereby contributing to medical services or drug development.

What is claimed is:

1. A node mapping method comprising the steps of:
searching a database storing interactions between nodes, and preparing a table which includes as elements node types, the number of connecting nodes to be connected to the nodes, and the number of end nodes to be connected to the nodes;
extracting from the table connecting nodes which are connected to a predetermined number of, or more, end nodes;
arranging the extracted connecting nodes onto a visualization space at a distance from each other, the distance being not less than a predetermined distance in accordance with the number of connecting nodes existing therebetween;
arranging the remaining connecting nodes onto the visualization space;
computing arrangement of the end nodes in the visualization space; and
adjusting the distance between the connecting nodes so that the end nodes do not overlap.

2. The node mapping method according to claim 1, wherein the connecting nodes are arranged on lattice points constituting the visualization space.

3. The node mapping method according to claim 1, wherein the nodes represent proteins.

4. The node mapping method according to claim 1, wherein the visualization space is a two-dimensional regular lattice.

5. A network visualization method comprising the steps of:
extracting, from a table which includes as elements node types, the number of connecting nodes to be connected to the nodes, and the number of end nodes to be connected to the nodes, connecting nodes which are connected to a predetermined number of, or more, end nodes;
arranging the extracted connecting nodes onto a visualization space at a distance from each other, the distance being not less than a predetermined distance in accordance with the number of connecting nodes existing therebetween;
arranging the remaining connecting nodes onto the visualization space;
computing arrangement of the end nodes in the visualization space;
adjusting the distance between the connecting nodes so that the end nodes do not overlap; and
screen-visualizing line segments which represent the connections between mutually connected nodes.

6. The network visualization method according to claim 5, wherein the connecting nodes are arranged on lattice points constituting the visualization space.

7. The network visualization method according to claim 5, wherein the nodes represent proteins.

8. The network visualization method according to claim 5, wherein the visualization space is a two-dimensional regular lattice.

9. A method for screening a regulatory substance, comprising the steps of:
extracting, from a table which includes as elements node types, the number of connecting nodes to be connected to the nodes, and the number of end nodes to be connected to the nodes, connecting nodes which are connected to a predetermined number of, or more, end nodes;
arranging the extracted connecting nodes onto a visualization space at a distance from each other, the distance being not less than a predetermined distance in accordance with the number of connecting nodes existing therebetween;
arranging the remaining connecting nodes onto the visualization space;
computing arrangement of the end nodes in the visualization space;
adjusting the distance between the connecting nodes so that the end nodes do not overlap;
screen-visualizing line segments which represent the connections between mutually connected nodes; and
screening the regulatory substance which regulates an interaction between the nodes on the basis of the screen-visualized information.

10. The screening method according to claim 9, wherein the regulatory substance is a substance which facilitates or attenuates the interaction.
